(19) United States
(12) Patent Application Publication
(10) Pub. No.: US 2008/0153901 A1
(43) Pub. Date: Jun. 26, 2008

(54) Avermectin/benzoyl peroxide compositions for treating afflictions of the skin, e.g., rosacea

(75) Inventors: Alexandre Kaoukhov, Juan Les Pins (FR); Colette Pernin, Nice (FR)

Correspondence Address: BUCHANAN, INGERSOLL & ROONEY PC, POST OFFICE BOX 1404, ALEXANDRIA, VA, 22313-1404, US

(73) Assignee: GALDERMA S.A., CHAM (CH)

(21) Appl. No.: 12/000,181

(22) Filed: Dec. 10, 2007

Related U.S. Application Data
(63) Continuation of application No. PCT/FR2006/001300, filed on Jun. 8, 2006.

(30) Foreign Application Priority Data
Jun. 10, 2005 (FR) 0505917

(51) Int. Cl. A61K 31/351; A61K 31/235; C07D 493/10

(52) U.S. Cl. 514/450; 514/544; 549/343

(57) ABSTRACT

The invention relates to a pharmaceutical composition, especially a dermatological composition, containing, in a physiologically acceptable medium, at least one compound of the avermectin family, and benzoyl peroxide, and also to its use for the manufacture of a medicament for treating skin conditions, especially rosacea.

The present invention relates to a pharmaceutical composition, and especially a dermatological composition, for treating skin conditions, and especially for treating rosacea (formerly known as acne rosacea). In particular, the invention relates to a pharmaceutical composition, especially a dermatological composition, comprising, in a physiologically acceptable medium, at least one compound of the avermectin family and benzoyl peroxide.

Rosacea is a chronic inflammatory dermatitis that mainly affects the median part of the face and the eyelids of certain adults. It is characterized by telangiectatic erythema, dryness of the skin, papules and pustules.

Conventionally, rosacea develops in adults between the ages of 30 and 50; it more frequently affects women, although the condition is generally more severe in men.

Despite its former name, acne rosacea is not a condition of the pilosebaceous follicles like juvenile acne, but a primitively vascular condition whose inflammatory stage lacks the cysts and comedones characteristic of common acne.

The aetiology of rosacea is still poorly understood, although many theories have been put forward. The most common hypothesis is based on the characteristic presence of the parasite Demodex folliculorum in the case of patients suffering from rosacea. This organism is absent in common acne. Other factors have been described as possibly contributing towards the development of rosacea, such as hormonal factors and especially endocrine factors, climatic and immunological factors, and bacterial factors via the presence of Helicobacter pylori, a bacterium associated with gastrointestinal disorders.

Rosacea develops in four stages over several years, in spasms aggravated by variations in temperature, alcohol, spices, exposure to sunlight and emotions. The various stages of the disease are the following:

Stage 1: stage of erythema episodes. The patients have erythrosis spasms due to the sudden dilation of the arterioles of the face, which then take on a congestive, red appearance. These spasms are caused by the emotions, meals and temperature changes.

Stage 2: stage of couperosis, i.e. of permanent erythema with telangiectasia. Certain patients also present oedema on the cheeks and the forehead.

Stage 3: inflammatory stage with appearance of inflammatory papules and pustules, but without affecting the sebaceous follicles and thus with absence of cysts and comedones.

Stage 4: rhinophyma stage. This late phase essentially affects men. The patients present a bumpy, voluminous red nose with sebaceous hyperplasia and fibrous reordering of the connective tissue.

Conventionally, rosacea is treated orally or topically with antibiotics such as tetracyclines, erythromycin or clindamycin, but also with vitamin A, salicylic acid, antifungal agents, steroids, metronidazole (an antibacterial agent) or with isotretinoin in severe cases, or even with anti-infectious agents such as benzoyl peroxide, or even with azelaic acid.

Benzoyl peroxide (or dibenzoyl peroxide) is known in the prior art for its anti-acne and keratolytic properties. Benzoyl peroxide has a bacteriostatic effect on Corynebacterium acnes, reduces the level of free fatty acids in sebaceous secretions and has strong oxidizing properties. Benzoyl peroxide is conventionally used in the treatment of common acne and, to a lesser extent, in the treatment of rosacea.

Patent U.S. Pat. No. 5,952,372 also describes a method for treating rosacea using ivermectin orally or topically in order to reduce and eliminate the parasite Demodex folliculorum present on the skin of patients.

Ivermectin belongs to the avermectin family, a group of macrocyclic lactones produced by the bacterium Streptomyces avermitilis (Reynolds JEF (Ed) (1993) Martindale. The Extra Pharmacopoeia. 29th Edition. Pharmaceutical Press, London).

The avermectins especially include ivermectin, invermectin, avermectin, abamectin, doramectin, eprinomectin and selamectin.

Ivermectin is known in the prior art for its antiparasitic and anthelmintic properties. The antiparasitic activity is thought to be due to the opening of a chlorine channel in the membrane of the neurons of the parasite under the effect of an increased release of the neuromediator GABA (gamma-aminobutyric acid), inducing neuromuscular paralysis that may lead to the death of certain parasites. Ivermectin also interacts with other chlorine channels, especially those dependent on the neuromediator GABA (gamma-aminobutyric acid).

Patent U.S. Pat. No. 6,133,310 describes the use of ivermectin in the treatment of rosacea in order to reduce and eliminate the parasite Demodex folliculorum present on the skin of patients.

However, these treatments have drawbacks such as irritation and intolerance phenomena, especially when they are used for a prolonged period. On the other hand, these treatments are only suppressive and not curative, acting especially on the pustulous spasms occurring during the inflammatory stage.

Taking the foregoing into account, there is thus a need for a composition that shows improved efficacy in the treatment of rosacea and that does not show the side effects described in the prior art. There is especially a need to produce a composition that imparts greater tolerance to the active principles, while at the same time reducing their side effects.

Accordingly, one subject of the present invention is a pharmaceutical composition, especially a dermatological composition, comprising, in a physiologically acceptable medium, at least one compound of the avermectin family and benzoyl peroxide. A subject of the invention is also the use of such a composition for the manufacture of a medicament for preventing and/or treating a skin condition.

According to a first aspect of the invention, a pharmaceutical composition, especially a dermatological composition, comprising at least one compound of the avermectin family and benzoyl peroxide may afford improved efficacy in the treatment of rosacea.

Such a composition may make it possible to substantially reduce the duration of the treatment and to obtain a greater reduction of the symptoms of rosacea.

Such a composition is especially intended for topical application.

The term “physiologically acceptable medium” means any medium that is compatible with the skin, mucous membranes and/or the integuments.

The invention and the advantages resulting therefrom will be understood more clearly on reading the description of the non-limiting embodiments that follow.

The compounds of the avermectin family that may be used according to the present invention especially include invermectin, ivermectin, avermectin, abamectin, doramectin, eprinomectin and selamectin. The compound of the avermectin family is preferentially ivermectin.

In the compositions according to the invention, the said compound of the avermectin family is present in concentrations of between 0.001% and 10% by weight and preferably between 0.01% and 5% by weight relative to the total weight of the composition.

In the compositions according to the invention, the benzoyl peroxide is present in concentrations of between 0.01% and 30% by weight and preferably between 0.1% and 20% by weight and particularly preferably between 1% and 10% by weight relative to the total weight of the composition.

Throughout the present text, unless otherwise specified, it is understood that when concentration ranges are given, they include the upper and lower limits of the said range.

Advantageously, the compositions of the invention comprise, besides at least one compound of the avermectin family and benzoyl peroxide, at least one other therapeutic active agent capable of increasing the efficacy of the treatment. Non-limiting examples of such agents that may be mentioned include antibiotics, antibacterial agents, antiviral agents, antiparasitic agents, antifungal agents, anaesthetics, analgesics, antiallergic agents, retinoids, free-radical scavengers, antipruritic agents, keratolytic agents, anti-seborrhoeic agents, antihistamines, sulfides, and immunosuppressant or antiproliferative products, or a mixture thereof.

The compositions according to the invention may also comprise any adjuvant usually used in cosmetics and dermatology that is compatible with the said compound of the avermectin family and benzoyl peroxide. Mention may be made especially of chelating agents, antioxidants, sunscreens, preserving agents, fillers, electrolytes, humectants, dyes, common mineral or organic acids or bases, fragrances, essential oils, cosmetic active agents, moisturizers, vitamins, essential fatty acids, sphingolipids, self-tanning compounds, calmatives and skin-protecting agents, pro-penetrating agents and gelling agents, or a mixture thereof. These adjuvants, and the concentrations thereof, should be such that they do not harm the advantageous properties of the mixture according to the invention. These additives may be present in the composition in a proportion of from 0 to 20% by weight and preferably from 1% to 10% by weight relative to the total weight of the composition.

Examples of preserving agents that may be mentioned include benzalkonium chloride, phenoxyethanol, benzyl alcohol, diazolidinylurea and parabens, or mixtures thereof.

Humectants that may be mentioned in particular include glycerol and sorbitol.

Examples of chelating agents that may be mentioned include ethylenediaminetetraacetic acid (EDTA) and also derivatives or salts thereof, dihydroxyethylglycine, citric acid and tartaric acid, or mixtures thereof.

Pro-penetrating agents that may be mentioned in particular include propylene glycol, dipropylene glycol, propylene glycol dipelargonate, lauryl glycol and ethoxydiglycol.

The compositions according to the invention are useful for treating and/or preventing rosacea.

According to a first embodiment of the invention, the use of the composition is intended for the manufacture of a medicament for treating the skin and preferably for treating rosacea, common acne and seborrhoeic dermatitis and particularly preferably for treating rosacea.

The invention also relates to the use of at least one compound of the avermectin family and benzoyl peroxide for the preparation of a pharmaceutical composition, and especially a dermatological composition, for preventing and/or treating a skin condition. In this use, the composition is as defined above.

The composition according to the invention is a pharmaceutical composition, and especially a dermatological composition, which may be in any galenical form conventionally used for topical application and especially in the form of aqueous gels, and aqueous or aqueous-alcoholic solutions. By addition of a fatty or oily phase, it may also be in the form of dispersions of the lotion or serum type, emulsions of liquid or semi-liquid consistency of the milk type obtained by dispersing a fatty phase in an aqueous phase (O/W) or conversely (W/O), or suspensions or emulsions of soft, semi-liquid or solid consistency of the cream, gel or ointment type, or alternatively multiple emulsions (W/O/W or O/W/O), microemulsions, microcapsules, microparticles or vesicular dispersions of ionic and/or nonionic type, or wax/aqueous phase dispersions. These compositions are prepared according to the usual methods.

When the composition is in emulsion form, the proportion of the oily phase of the emulsion may range, for example, from 5% to 80% by weight and preferably from 5% to 50% by weight relative to the total weight of the composition. The oils, emulsifiers and co-emulsifiers used in the composition in emulsion form are chosen from those conventionally used in cosmetics or dermatology. The emulsifier and the co-emulsifier are generally present in the composition in a proportion ranging from 0.3% to 30% by weight and preferably from 0.5% to 20% by weight relative to the total weight of the composition. The emulsion may also contain lipid vesicles.

As fatty substances that may be used in the invention, it is possible to use oils and especially mineral oils (liquid petroleum jelly), oils of plant origin (avocado oil or soybean oil), oils of animal origin (lanolin), synthetic oils (perhydrosqualene), silicone oils (cyclomethicone) and fluoro oils (perfluoropolyethers). Fatty alcohols such as cetyl alcohol, fatty acids, waxes and gums, in particular silicone gums, may also be used as fatty substances.

As emulsifiers and co-emulsifiers that may be used in the invention, examples that may be mentioned include fatty acid esters of polyethylene glycol such as PEG-100 stearate, PEG-50 stearate and PEG-40 stearate; fatty acid esters of polyols such as glyceryl stearate, sorbitan tristearate and the oxyethylenated sorbitan stearates available under the trade name Tween 20 or Tween 60, for example; and mixtures thereof.

Non-limiting examples of gelling agents that may be mentioned include the polyacrylamide family such as the sodium acryloyldimethyltaurate copolymer/isohexa-decane/polysorbate 80 mixture sold under the name Simulgel™ 600 by the company SEPPIC, the polyacrylamide/C13-14 isoparaffin/Laureth-7 mixture, for instance the product sold under the name Sepigel 305™ by the company SEPPIC, the family of acrylic polymers coupled to hydrophobic chains, such as the PEG-150/decyl/SMDI copolymer sold under the name Aculyn 44™ (polycondensate comprising at least, as components, a polyethylene glycol containing 150 or 180 mol of ethylene oxide, decyl alcohol and methylenebis(4-cyclohexyl isocyanate) (SMDI), at 35% by weight in a mixture of propylene glycol (39%) and water (26%)), and the family of modified starches such as the modified potato starch sold under the name Structure Solanace™, or mixtures thereof.

The preferred gelling agents are derived from the polyacrylamide family, such as Simulgel 600™ or Sepigel 305™, or mixtures thereof.

The gelling agent as described above may be used in a concentration ranging from 0.1% to 15% and preferably from 0.5% to 5%.

1. Pharmaceutical composition, especially a dermatological composition, characterized in that it comprises, in a physiologically acceptable medium, at least one compound of the avermectin family and benzoyl peroxide.

2. Composition according to claim 1, characterized in that the compound of the avermectin family is chosen from invermectin, ivermectin, avermectin, abamectin, doramectin, eprinomectin and selamectin.

3. Composition according to claim 1 or 2, characterized in that the compound of the avermectin family is ivermectin.

4. Composition according to any one of claims 1 to 3, characterized in that the compound of the avermectin family represents between 0.001% and 10% by weight and preferably between 0.01% and 5% by weight relative to0 the total weight of the composition.

5. Composition according to any one of claims 1 to 4, characterized in that the concentration of benzoyl peroxide is between 0.01% and 30% by weight and preferably between 0.1% and 20% by weight and particularly preferably between 1% and 10% by weight relative to the total weight of the composition.

6. Composition according to any one of claims 1 to 5, characterized in that it is for topical application.

7. Composition according to any one of claims 1 to 6, characterized in that it also contains at least one active agent chosen from the group comprising antibiotics, antibacterial agents, antiviral agents, antiparasitic agents, antifungal agents, anaesthetics, analgesics, antiallergic agents, retinoids, free-radical scavengers, antipruritic agents, keratolytic agents, anti-seborrhoeic agents, antihistamines, sulfides, and immunosuppressant or antiproliferative products.

8. Composition according to any one of claims 1 to 7, characterized in that it also contains at least one additive chosen from the group comprising chelating agents, antioxidants, sunscreens, preserving agents, fillers, electrolytes, humectants, dyes, common mineral or organic acids or bases, fragrances, essential oils, cosmetic active agents, moisturizers, vitamins, essential fatty acids, sphingolipids, self-tanning compounds, calmatives and skin-protecting agents, pro-penetrating agents and gelling agents, or a mixture thereof.

9. Use of at least one compound of the avermectin family and benzoyl peroxide according to any one of claims 1 to 8 for the manufacture of a medicament for preventing and/or treating a skin condition.

10. Use according to claim 9, characterized in that the medicament is intended for treating and/or preventing rosacea, common acne and/or seborrhoeic dermatitis.

11. Use according to claim 9 or 10, characterized in that the medicament is intended for treating and/or preventing rosacea.
